(19) United States
(12) Patent Application Publication
(10) Pub. No.: US 2003/0088039 A1
(43) Pub. Date: May 8, 2003

(54) Composition

(76) Inventors: Anke Glumer, Braunschweig (DE); Klaus Buchholz, Braunschweig (DE); Shukun Yu, Malmoe (SE)

Correspondence Address: FROMMER LAWRENCE & HAUG, 745 FIFTH AVENUE- 10TH FL., NEW YORK, NY, 10151, US

(21) Appl. No.: 10/138,800

(22) Filed: May 3, 2002

Related U.S. Application Data
(63) Continuation-in-part of application No. PCT/IB00/01574, filed on Oct. 12, 2000.

(30) Foreign Application Priority Data
Nov. 4, 1999 (GB) 9926175.2
Jan. 27, 2000 (GB) 0001939.8

(51) Int. Cl. C08F 2/00; C07H 1/00

(52) U.S. Cl. 526/238.2; 536/1.11

(57) ABSTRACT

The present invention provides a composition comprising at least two different polymerisable monomers, (i) the first monomer unit is a polymerisable derivative of anhydrofructose; and (ii) the second monomer unit is other than a polymerisable derivative of anhydrofructose.

### CROSS REFERENCE TO RELATED APPLICATIONS

This application is a Continuation-in-Part of PCT/IB00/01574, filed Oct. 12, 2000, designating the U.S., published May 10, 2001 as WO 01/32728 and claiming priority from GB 9926175.2 filed Nov. 4, 1999 and GB 0001939.8 filed Jan. 27, 2000. All of the above-mentioned applications, as well as all documents cited herein and documents referenced or cited in documents cited herein, are hereby incorporated herein by reference.

### SUMMARY OF THE INVENTION

The present invention relates to copolymers of anhydrofructose derivatives and at least one further monomer unit. In particular, the present invention relates copolymers of 3,6-di-O-acetyl-1,5-anhydro-4-deoxy-D-glycerohex-3-enopyranose-2-ulose, an acylated derivative of 1,5-Anhydro-D-fructose and vinylacetate, or vinylbutylether.

### BACKGROUND

Polymers, including homopolymers and copolymers are extensively used in industry. Typically, the polymers are based on petroleum products and derivatives. An example of such a well know polymer is polystyrene. The synthesis of polystyrene is illustrated in Scheme 1 of FIG. 1.

Petroleum based polymers are typically not biodegradable or biocompatible. As such the use thereof has become less acceptable to consumers at least and biodegradable or biocompatible alternatives have been sought.

The prior discloses some attempts to synthesise biopolymers. For example U.S. Pat. No. 5,618,933 and U.S. Pat. No. 5,854,030 disclose the process of Scheme 2 wherein a sugar such a glucose is enzymatically derivatised to carry a polymerisable group. The polymerisable group is then polymerised to provide a homopolymer. These processes attempt to synthesise a well-defined semi-biopolymer. In these syntheses, in place of the phenyl group as the pendant group of Scheme 1, the pendant group in these semi-biopolymers are sugar residues. U.S. Pat. No. 5,618,933 and U.S. Pat. No. 5,854,030 report that the presence of the sugar residues results in the polymer being a hydrogel. It is taught that the hydrogel can absorb water up to 1100 times of its own weight.

The prior art also provides teachings of polymers comprising unsaturated sugars in the backbone of the polymer (see Buchholz et al., 1994. Synthesis of new “Saccharide polymers” from unsaturated monosaccharides. In: Carbohydrates as Organic Raw Materials III (edited by van Bekkum H., Röper H., and A. G. J. Voragen). VCH Publishers, Inc., New York (USA), ISBN 3-527-30079-1 AND WO-A-99/00436). However, a problem with these syntheses and the disclosed polymers has been the high cost of the unsaturated sugars incorporated in the polymer. For example the preparation of an unsaturated glucose derivative requires a multi-step process. As a result of this, the price of the unsaturated sugars and therefore the cost of the polymers prepared with the sugar derivatives are high.

### BRIEF DESCRIPTION OF THE FIGURES

FIG. 1: Scheme 1 is a schematic drawing of the synthesis of polystyrene. Scheme 2 is a schematic drawing of the synthesis of a sugar-based polymer wherein the sugar units are hanging on the backbone of the polymer.

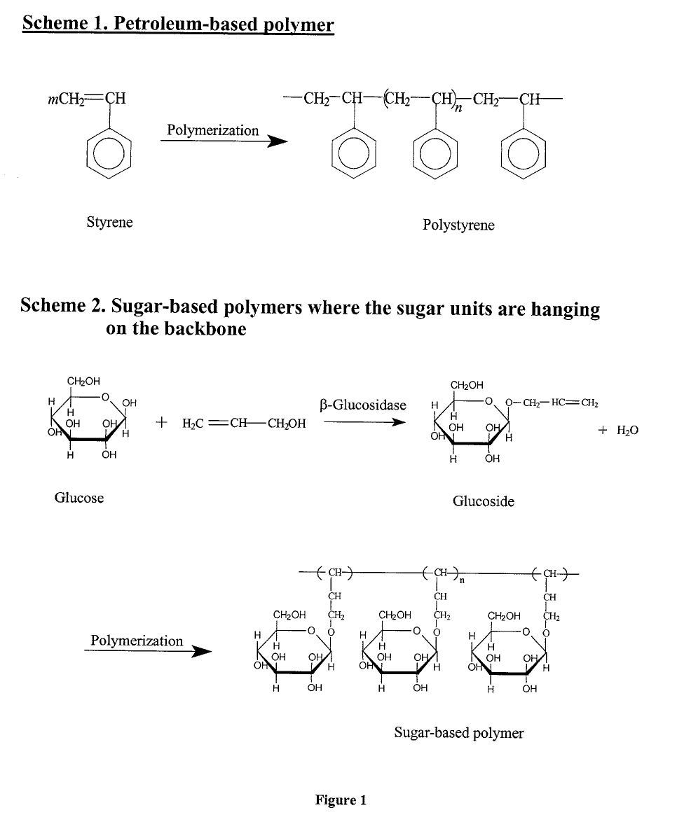

FIG. 2: Scheme 3 is a schematic drawing of the synthesis of a sugar-based polymer wherein the sugar units are not in the backbone.

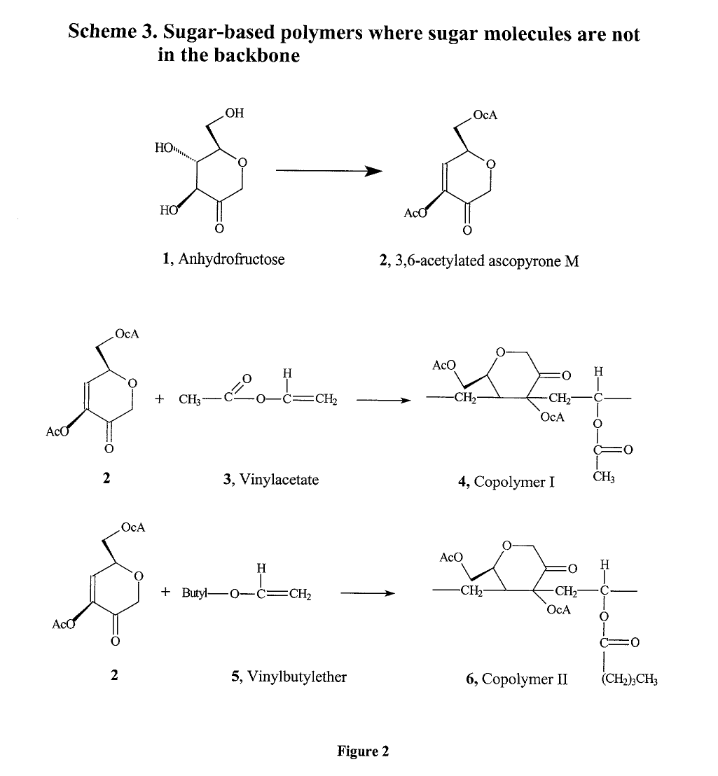

### DETAILED DESCRIPTION

The present invention addresses the problems of the prior art.

In a first aspect the present invention provides a composition comprising at least two different polymerisable monomers, (i) the first monomer unit is a polymerisable derivative of anhydrofructose; and (ii) the second monomer unit is other than a polymerisable derivative of anhydrofructose.

It is noted that a polymerisable derivative or polymerisable monomer is a derivative or monomer having a suitable electron density, e.g., saturation or double and/or triple bond(s), such that the derivative or monomer is capable of polymerising, e.g., in the presence of an initiator such as a chemical initiator UV light.

Preferably at least one ring of the polymerisable derivative of anhydrofructose is unsaturated.

More preferably the polymerisable derivative of anhydrofructose is of General Formula A

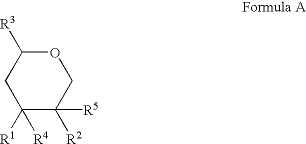

or a derivative thereof

wherein R¹ and R² are independently selected from —OH, ═O

wherein R³ is a substituent comprising an —OH group; and

wherein R⁴ and R⁵ are independently selected from —OH, ═O or represent a bond with an adjacent atom on the ring of the cyclic compound, wherein at least one of R⁴ and R⁵ represent a bond with an adjacent atom on the ring of the cyclic compound.

Preferably R³ of General Formula A is or comprises an —CH₂OH group.

In this respect, preferably the first monomer unit is selected from Ascopyrone M, Ascopyrone P, Ascopyrone T₂ and derivatives thereof.

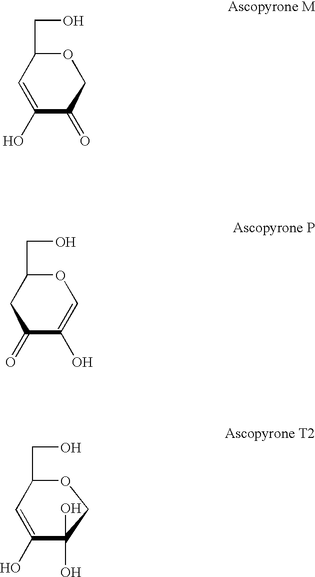

Preferably the first monomer unit is protected. Preferably, the first monomer unit is protected by an acyl group or a benzoyl group (C₆H₅CO—). The first monomer unit preferably comprises an acyl group. It is well understood that the term acyl means a group R—C(═O)—.

In a preferred aspect at least one ring of the polymerisable derivative of anhydrofructose is of General Formula A (more preferably is selected from Ascopyrone M, Ascopyrone P, and Ascopyrone T₂) and the polymerisable derivative comprises an acyl group. Thus in a preferred aspect the first monomer unit is of the General Formula B

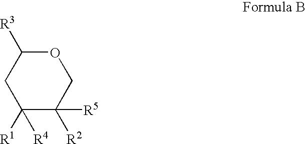

or a derivative thereof

wherein R¹ and R² are independently selected from —OH, ═O

wherein R³ is a substituent comprising an —OH group; and

wherein R⁴ and R⁵ are independently selected from —OH, ═O or represent a bond with an adjacent atom on the ring of the cyclic compound, wherein at least one of R⁴ and R⁵ represent a bond with an adjacent atom on the ring of the cyclic compound, and

wherein at least one of R¹ to R⁵ is an acyl group.

More preferably the first monomer unit is of the formula

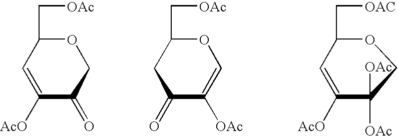

Yet more preferably, the first monomer unit is of the formula

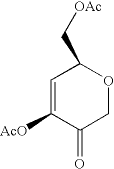

The second monomer unit may be any suitable monomer providing it is different ot the first monomer unit. Preferably the second monomer unit comprises a vinyl group. More preferably, the second monomer unit is selected from vinylacetate, vinylbutylether, styrene, derivatives and mixtures thereof.

In a further aspect the present invention provides a polymerisation product of a composition as described herein.

In a particularly preferred aspect the present invention provides a polymer comprising the unit

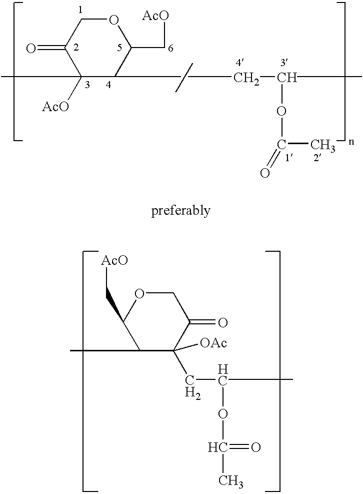

The polymer can comprise at least one unit or multiples thereof (wherein the multiple can be multiples of whole numbers, fractions, or whole numbers plus fractions, i.e. the multiples can be ½, 1, 1½, . . . 300, 300½, 301 . . . ) wherein n is advantageously an integer greater than or equal to one, and advantageously can be up to and including the integer which provides an average moleuclar weight of 21000 g/mol, and any integer, whole number or fraction therebetween; and, of course n may even be greater.

In a particularly preferred aspect the present invention provides a polymer comprising the unit

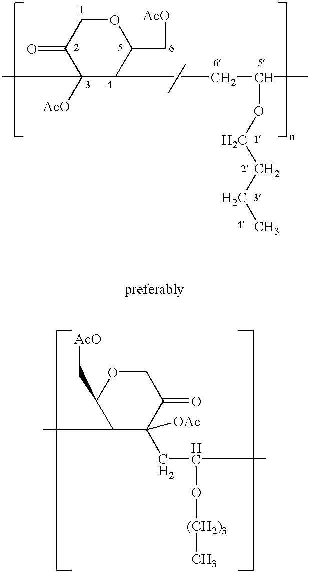

The polymer can comprise at least one unit or multiples thereof (wherein the multiple can be multiples of whole numbers, fractions, or whole numbers plus fractions, i.e. the multiples can be ½, 1, 1½, . . . 300, 300½, 301 . . . ) wherein n is advantageously an integer greater than or equal to one, and advantageously can be up to and including the integer which provides an average moleuclar weight of 950000 g/mol, and any integer, whole number or fraction therebetween; and, of course n may even be greater.

In further aspects the present invention provides a polymer comprising the unit selected from

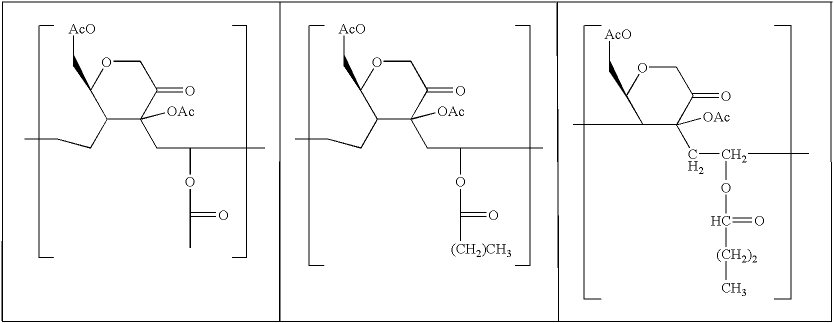

Anhydrofructose may be prepared for use in the present invention by any available means. In one aspect anhydrofructose may be produced directly from starch as described in S. Yu et al. (1999). α-1,4-Glucan lyases producing 1,5-anhydro-D-fructose from starch and glycogen have sequence similarity to alpha-glucosidases. Biochim. Biophys. Acta. 1433(1-2):1-15.

When the first monomer unit is 3,6-Di-O-acetyl-1,5-anhydro-4-deoxy-D-glycerohex-3-enopyranose-2-ulose (3,6-acetylated ascopyrone M), the monomer may be prepared from anhydrofructose according to Freimund, S. and Köpper S. 1998. Dimeric structures of 1,5-anhydro-D-fructose. Carbohydr. Res. 308: 195-200, or Andersen, S. M. et al. Structure of 1,5-anhydro-D-fructose: X-ray analysis of crystalline acetylated dimeric forms. J. Carbohydr. Chem. 17(7):1027-1035, 1998.

In a preferred aspect of the present invention a polymer may then be provided by the copolymerisation of 3,6-acetylated ascopyrone M with a co-monomer, such as vinylacetate, vinylbutylether. The copolymers yielded by this polymerisation are illustrated in Scheme 3. The novel sugar-based copolymers provided are Copolymer I and Copolymer II, respectively.

The polymerisation of the present invention may be carried out using polymerisation conditions well known to a person skilled in the art. The method used to prepare Copolymer I and Copolymer II is that described by Buchholz et al., and WO-A-99/00436.

When the first monomer unit of the composition of the present invention comprises an acyl group, the acyl group may be hydrolysed after polymerisation of the composition. In this aspect, it is possible to modify the hydrophobicity of the polymer by this hydrolysis. For example the acetyl groups on Copolymer I and Copolymer II may be hydrolysed to form a series of copolymers with different degree of acetylation and therefore different degree of hydrophobicity.

The polymers of the present invention may be used in any application where provision of a hydrogel is required. For example the polymer may be used in absorbent products such as nappies (diapers), both for children and adults, including but not limited to disposable undergarments, disposable briefs, underpads, adult pull-ups, guards for men, as well as in packaging materials, drug delivery polymers, bandages, medical devices such as ophthalmic devices, feminine hygiene or sanitary products, and in a variety of other commercial applications. The present polymers may be particularly advantageous as biocompatible polymers are provided. Such polymers may be used for the preparation of topically applied materials such as cosmetics, dressings or pharmaceutical compositions which do not irritate the skin.

### EXAMPLES

Four copolymerisations were performed. Acetylated anhydrofructose derivative (AnF) was copolymerised with each of vinylacetate (Vac) and vinylbutylether (VBE). Each polymerisation was carried out in solution and in substance

### Example 1

Preparation of Poly(acetyl-Anhydro-fructose)-co-(Vinylacetat)

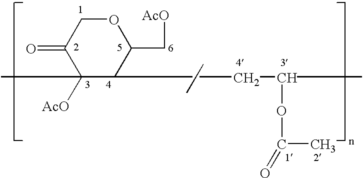

0.5 g (0.00218 mol) 3,6-Di-O-acetyl-1,5-anhydro-4-deoxy-D-glycero-hex-3-enos-2-ulopyranose was placed in a pressure stable polymerisation reactor which was equipped with a thermometer, heating, dosing device and argon inlet and outlet.

To this liquid syrup were mixed 0.242 mL (0.226 g) vinylacetate (Saccharide:Vinylacetate (1:1)) and 8 mg dibenzoylperoxide. The mixture was degassed by the freeze-thaw method. The mixture was polymerised at 80° C. in the sealed reactor for 48 hours. After the reaction has finished a pale yellow solid with 14 wt. % yield was obtained. The analysis by GPC-MALLS gave a weight average molecular weight of 210000 g/mol.

¹H-NMR (400.1 MHz; CDCl₃): δ=1.6-2.3 (11H, 9H-acetyl and H-4′); 2.4-3.2 (1H, perhaps H-4); 3.2-5.2 (6H, H-1/4/5/6 and H-3′).

¹³C-NMR (100.6 MHz, CDCl₃): δ=20-22 (2xCH₃-acetyl and C-2′); 37-38 (C-4′); 52-54 (C-4); 63-80 (C-1/3/5/6 and C-3′); 165-173 (—COO—acetyl and C-1′); 200 (C-2).

FT-IR (KBr): \tilde{ν} (cm⁻¹) =2962 (CH₂,CH₃); 1743 (C═O); 1432 (C—H, acetyl); 1234 (CH₂-Def., Ester); 1045 (C—O-Valenz).

[α]_{D}²⁰=−13.1 (CHCl₃, c=0.655 g/100 mL).

| | | | |
|---|---|---|---|
| (C₁₀H₁₂O₆)_{0.5}(C₄H₆O₂)_{0.5} (314.29)ₙ: | calculated: | C: 53.50 | H: 5.73 |
| | found: | C: 53.13 | H: 5.59 |

### Example 2

Example 1 was repeated in solution (toluene). Traces of polymer were detectable by TLC.

### Example 3

Preparation of Poly(acetyl-Anhydro-fructose)-co-(Vinylbutylether)

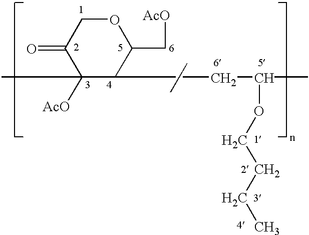

0.5 g (0.00218 mol) 3,6-Di-O-acetyl-1,5-anhydro-4-deoxy-D-glycero-hex-3-enos-2-ulopyranose was placed in a pressure stable polymerisation reactor which was equipped with a thermometer, heating, dosing device and argon inlet and outlet.

To this liquid syrup were mixed 0.281 mL (0.286 g) vinylbutylether (Saccharide:Vinylbutylether (1:1)) and 8 mg dibenzoylperoxide and the mixture was degassed by the freeze-thaw method. The mixture was polymerised at 80° C. in the sealed reactor for 48 hours. After the reaction has finished a dark yellow solid with 11 wt-% yield was obtained. The analysis by GPC-MALLS gave a weight average molecular weight of 950000 g/mol.

¹H-NMR (400.1 MHz; CDCl₃): δ0.8-1.0 (3H, H-4′); 1.2-1.6 (4H, H-2′/3′); 1.7-2.3 (8H, CH₃, acetyl, H-6′); 2.4-3.7 (4H, H-1′/5′ and H-4); 3.8-5.1 (5H, H-1/5/6).

¹³C-NMR (100.6 MHz, CDCl₃): δ=14.0 (C-4′); 19 (C-3′); 21 (C-acetyl); 32 (C-2′); 39 C-6′); 41 (C-4); 45 (C-1′); 50-52 (C-5′); 65 (C-6); 70-80 (C-1/3/5/6); 168-171 (—COO—, acetyl); 200 (C-2).

FT-IR (KBr): \tilde{ν} (cm⁻¹)=2962; 2938; 2875 (CH₂,CH₃); 1745 (C═O); 1453; 1436 (C—H, acetyl); 1371;1234 (CH₂-Def., C—O, Ester); 1177, 1068, 1027 (C—O-Valenz, Ether).

[α]_{D}²⁰=−28.11 (CHCl₃, c=1.11 g/100 mL).

Characterisation of

Poly(acetyl-Anhydro-fructose)-co-(Vinylacetat) (AnF-Vac) and

Poly(acetyl-Anhydro-fructose)-co-(Vinylbutylether) (AnF-VBE)

| | | | [α]_{D}^{20b)} | Polymer | |
|---|---|---|---|---|---|
| | \overline{M}w^{a)} | Tg | (deg · cm² · | Composition | Yield^{c)} |
| Polymer | (g/mol) | (° C.) | dag⁻¹) | An:Comon | (wt %) |
| AnF-Vac | 2.10 · 10⁵ | ˜63 | −13.1 | 58:42 | 14 |
| AnF-VBE | 9.50 · 10⁵ | 81.6 | −28.1 | 40.5:59.5 | 11 |

Polymerisation conditions: Temperature: 80° C.; Reactiontime: 48 h; Concentration of Monomers were 0.00218 mol; Initiator: Dibenzoylperoxide 1 mol %.
^{a)}Weight average molecular weights determined by means of GPC online with MALLS.
^{b)}Specific optical rotations, measured in CHCl₃ solution.
^{c)}Isolated products.

### Example 4

Example 4 was repeated in solution (toluene). Traces of polymer were detectable by TLC.

### Example 5

Hydrolysis of polymers—the solid polymer of the above Examples was pulverised by ball milling, dissolved in 1-2N sodium hydroxide solution and stirred for a few days; if necessary the solution was warmed to 50° C. until all polymer was dissolved. The solvent was concentrated and the pH value was adjusted to 10-11. The solution was desalted by dialysis (dialysing membrane MWCO 3500). The desalted solution was freeze dried.

### Example 6

The polymer of Example 1 is incorporated in to the liner of a child's diaper or into the adult equivalent of a diaper for conditions such as incontinence. The absorption capability of the nappy is tested by dispensing 50 ml of water on the inner surface of the diaper. The water is absorbed by the polymer of the present invention and on application of pressure to the surface of the diaper the water is not released from the polymer.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. A composition comprising at least two different polymerisable monomers,
(i) the first monomer unit is a polymerisable derivative of anhydrofructose; and
(ii) the second monomer unit is other than a polymerisable derivative of anhydrofructose.

2. The composition of claim 1, wherein at least one ring of the anhydrofructose derivative is unsaturated.

3. The composition of claim 1, wherein the polymerisable derivative of anhydrofructose is of Formula A 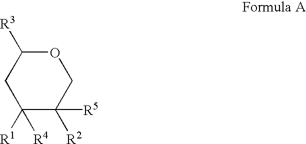
or a derivative thereof
wherein R¹ and R² are independently selected from —OH, ═O
wherein R³ is a substituent comprising an —OH group; and
wherein R⁴ and R⁵ are independently selected from —OH, ═O or represent a bond with an adjacent atom on the ring of the cyclic compound, wherein at least one of R⁴ and R⁵ represent a bond with an adjacent atom on the ring of the cyclic compound.

4. The composition of claim 3, wherein R³ is or comprises an —CH₂OH group.

5. The composition of claim 1, wherein the first monomer unit is selected from Ascopyrone M, Ascopyrone P, Ascopyrone T₂ and derivatives thereof.

6. The composition of claim 1, wherein the first monomer unit is an acylated or benzoylated derivative of anhydrofructose.

7. The composition of claim 1, wherein the first monomer unit is of the Formula B 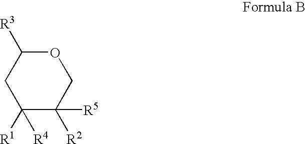
or a derivative thereof
wherein R¹ and R² are independently selected from —OH, ═O
wherein R³ is a substituent comprising an —OH group; and
wherein R⁴ and R⁵ are independently selected from —OH, ═O or represent a bond with an adjacent atom on the ring of the cyclic compound, wherein at least one of R⁴ and R⁵ represent a bond with an adjacent atom on the ring of the cyclic compound, and
wherein at least one of R¹ to R⁵ is an acyl group.

8. The composition of claim 1, wherein the first monomer unit is of the formula selected from any one of the following:

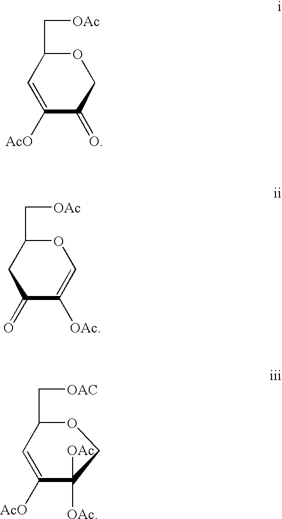

9. The composition of claim 8, wherein the first monomer unit is of the formula

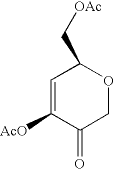

10. The composition of claim 1, wherein the second monomer unit comprises a vinyl group.

11. The composition of claim 1, wherein the second monomer unit is selected from vinylacetate, vinylbutylether, styrene, derivatives and mixtures thereof.

12. A polymerisation product of a composition as defined in claim 1.

13. A polymer comprising the unit

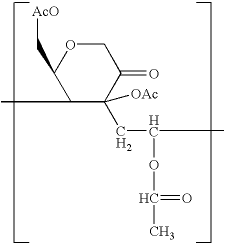

14. A polymer comprising the unit

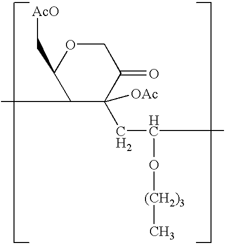

15. A polymer which is the polymerization product of claim 12.

16. A method of preparing a polymer comprising polymerising a first monomer unit wherein the first monomer unit is a polymerisable derivative of anhydrofructose; and polymerising a second monomer unit, wherein the second monomer unit is other than a polymerisable derivative of anhydrofructose.

17. The method of claim 16, wherein at least one ring of the anhydrofructose derivative is unsaturated.

18. The method of claim 16, wherein the polymerisable derivative of anhydrofructose is of Formula A 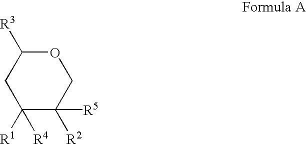
or a derivative thereof
wherein R¹ and R² are independently selected from —OH, ═O
wherein R³ is a substituent comprising an —OH group; and
wherein R⁴ and R⁵ are independently selected from —OH, ═O or represent a bond with an adjacent atom on the ring of the cyclic compound, wherein at least one of R⁴ and R⁵ represent a bond with an adjacent atom on the ring of the cyclic compound.

19. The method of claim 18, wherein R³ is or comprises an —CH₂OH group.

20. The method of claim 16, wherein the first monomer unit is selected from Ascopyrone M, Ascopyrone P, Ascopyrone T₂ and derivatives thereof.

21. The method of claim 16, wherein the first monomer unit is an acylated or benzoylated derivative of anhydrofructose.

22. The method of claim 16 wherein the first monomer unit is of the Formula B 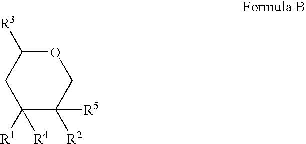
or a derivative thereof
wherein R¹ and R² are independently selected from —OH, ═O
wherein R³ is a substituent comprising an —OH group; and
wherein R⁴ and R⁵ are independently selected from —OH, ═O or represent a bond with an adjacent atom on the ring of the cyclic compound, wherein at least one of R⁴ and R⁵ represent a bond with an adjacent atom on the ring of the cyclic compound, and
wherein at least one of R¹ to R⁵ is an acyl group.

23. The method of claim 16, wherein the first monomer unit is of the formula selected from any one of the following:

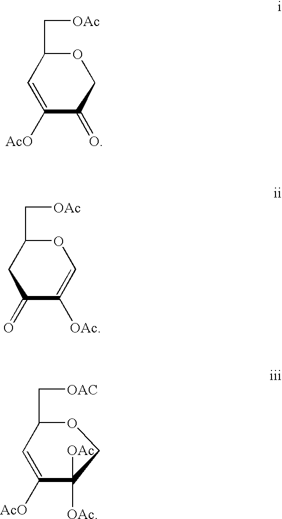

24. The method of claim 23, wherein the first monomer unit is of the formula

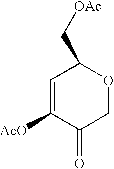

25. The method of claim 16, wherein the second monomer unit comprises a vinyl group.

26. The method of claim 16, wherein the second monomer unit is selected from vinylacetate, vinylbutylether, styrene, derivatives and mixtures thereof.
